(19) United States
(12) Patent Application Publication
(10) Pub. No.: US 2026/0026676 A1
(43) Pub. Date: Jan. 29, 2026

(54) BATTERY PACKS FOR MULTIPLE TYPES OF VIDEO LARYNGOSCOPES

(71) Applicant: Covidien LP, Mansfield, MA (US)

(72) Inventors: Jakob STRUIK, Hong Kong (HK); Alexandra HAUSE, Denver, CO (US)

(73) Assignee: Covidien LP, Mansfield, MA (US)

(21) Appl. No.: 19/270,743

(22) Filed: Jul. 16, 2025

Related U.S. Application Data
(60) Provisional application No. 63/675,570, filed on Jul. 25, 2024.

(51) Int. Cl. A61B 1/00; A61B 1/04; A61B 1/267

(52) U.S. Cl. CPC A61B 1/00032; A61B 1/00006; A61B 1/00057; A61B 1/00059; A61B 1/04; A61B 1/267

(57) ABSTRACT

The technology relates to a battery pack that can be used to provide power to multiple different types of laryngoscopes. The battery pack includes a processor and is configured to determine the type of medical device in which it is installed. The battery pack performs different operations depending on the type of medical device. For example, the battery pack provides different voltages to the medical devices depending on the type of medical device, and maintains end-of-life counters in the RFID tag in different manners. The battery pack includes a capacity gauge and is configured to measure its own battery capacity and store a corresponding remaining capacity value in the RFID tag for reading by the medical device.

### CROSS-REFERENCE TO RELATED APPLICATIONS

This application claims the benefit of U.S. Provisional Application No. 63/675,570, filed Jul. 25, 2024, entitled “Battery Packs for Multiple Types of Video Laryngoscopes,” which is incorporated herein by reference in its entirety.

### BACKGROUND

Medical devices such as video laryngoscopes may incorporate a battery power supply to enable them to be used without the limitations of a power cable. A battery pack may be installed in a handle or body of the medical device, for example.

It is with respect to this general technical environment that aspects of the present technology disclosed herein have been contemplated. Furthermore, although a general environment is discussed, it should be understood that the examples described herein should not be limited to the general environment identified herein.

### SUMMARY

This summary is provided to introduce a selection of concepts in a simplified form that are further described below in the Detailed Description. This summary is not intended to identify key features or essential features of the claimed subject matter.

Aspects disclosed herein include a battery pack configured to be installed in a video laryngoscope to provide power to the video laryngoscope. The battery pack includes a battery; a radio-frequency identification (RFID) tag; and a processor, where the processor is configured to cause the battery pack to: determine a type of the video laryngoscope; and perform one of: in accordance with a determination that the video laryngoscope is a first type of video laryngoscope, maintain a first end-of-life (EOL) counter by periodically updating the first EOL counter in the RFID tag based on a remaining capacity of the battery and power consumption information associated with the first type of video laryngoscope; or in accordance with a determination that the video laryngoscope is not the first type of video laryngoscope, maintain a second EOL counter by updating the second EOL counter in response to signals received from the video laryngoscope.

In examples, the battery pack includes a capacity gauge configured to measure the remaining capacity of the battery, where the processor is further configured to cause the battery to: in response to detecting a power up condition: determine, using the capacity gauge, the remaining capacity of the battery; and save a remaining capacity value to the RFID tag based on the remaining capacity of the battery. In examples, the battery pack is configured to: periodically determine, using the capacity gauge, the remaining capacity; and update the remaining capacity value based on the remaining capacity. In examples, the battery pack includes a voltage regulator, and the processor is further configured to cause the battery pack to perform one of: in accordance with the determination that the video laryngoscope is the first type of video laryngoscope, provide a first voltage to the video laryngoscope using the voltage regulator, wherein the first voltage is a fixed voltage; and in accordance with the determination that the video laryngoscope is the second type of video laryngoscope, bypass the voltage regulator to provide a second voltage to the video laryngoscope as a direct output from the battery. In examples, the processor is further configured to cause the battery pack to: in response to powering on, perform one of: in accordance with the determination that the video laryngoscope is a first type of video laryngoscope, provide a first power-on signal to the video laryngoscope at a first time; and in accordance with the determination that the video laryngoscope is a second type of video laryngoscope, provide a second power-on signal to the video laryngoscope at a second time. In examples, the remaining capacity is in units of milliamp hours. In examples, the processor is configured to cause the battery pack to determine the type of video laryngoscope by identifying an electrical characteristic of the video laryngoscope. In examples, the processor is configured to cause the battery pack to disable a power output of the battery pack based on a determination that the first EOL counter, the second EOL counter, or a combination of the first EOL counter and the second EOL counter has reached a threshold.

Aspects disclosed herein further include a system that includes a battery pack comprising a battery, a first processor, an RFID tag, a capacity gauge, and an activation switch, where the battery pack performs operations comprising: detecting, by the battery pack via the activation switch, a power up condition; in response to detecting the power up condition, measuring, using the capacity gauge, a remaining capacity of the battery; saving a remaining capacity value to the RFID tag based on the remaining capacity of the battery; receiving, at the RFID tag, a request to provide the remaining capacity value to a video laryngoscope comprising a second processor and a radio-frequency identification (RFID) tag reader; providing the remaining capacity value to the video laryngoscope, where the video laryngoscope performs operations comprising: generating, by the video laryngoscope based at least in part on the remaining capacity value and a power consumption associated with the video laryngoscope, a remaining minutes value; and displaying, on a display of the video laryngoscope, the remaining minutes value.

In examples, the operations performed by the video laryngoscope include: periodically send, using the RFID tag reader, a signal to the RFID tag to cause the RFID tag to update an end-of-life (EOL) counter in the RFID tag. In examples, the operations of the video laryngoscope include: periodically obtaining an updated remaining capacity value from the RFID tag, updating the remaining minutes value based on the updated remaining capacity value, and displaying the updated remaining minutes value. In examples, the operations performed by the battery pack include: periodically determining, using the capacity gauge, the remaining capacity; and updating the remaining capacity value based on the remaining capacity. In examples, the battery pack includes a voltage regulator, and the operations performed by the battery pack include: determining that the video laryngoscope is a particular type of video laryngoscope; and in accordance with the determination that the video laryngoscope is the particular type of video laryngoscope, providing a variable voltage to the video laryngoscope as an output from the battery pack. In examples, the second processor is configured to cause the battery pack to determine that the video laryngoscope is a first type of video laryngoscope by: measuring an electrical load of the video laryngoscope in milliamps, and determining that the electrical load exceeds a threshold electrical load.

Aspects disclosed herein further include battery pack for a video laryngoscope, the battery pack including: a radio-frequency identification (RFID) tag having access to a unit-capacity section, an energy-capacity section, a first end-of-life (EOL) counter, and a second EOL counter; a processor; and memory storing instructions that, when executed by the processor, cause the battery pack to perform operations including: while connected to a first type of video laryngoscope: receiving a read request, from the first type of video laryngoscope, for a charge capacity value from the unit-capacity section; receiving an update, from the first type of video laryngoscope, to the unit-capacity section indicating units of energy consumed by the first type of video laryngoscope; and based on the units of energy consumed by the first type of video laryngoscope, update the first EOL counter; and while connected to a second type of video laryngoscope: receiving a read request, from the second type of video laryngoscope, for a charge capacity value from the energy-capacity section; and receiving, from the second type of video laryngoscope, an update to the second EOL counter.

In examples, the operations further comprise: while charging: aggregating time of use from the first EOL counter and the second EOL counter to determine total lifetime use of the battery pack; and based on total lifetime of use, indicate at least one of an EOL warning or an EOL status. In examples, the EOL warning is indicated based on the total lifetime of use being between a first threshold and a second threshold, and the EOL warning indicates the battery is approaching end of life. In examples, the EOL status is indicated based on the total lifetime of use being above the second threshold, wherein the EOL status indicates that the battery has reached its end of life. In examples, the battery pack further includes a charge gauge, and the operations further include: measuring, by the charge gauge, a charge capacity of the battery pack; and updating the unit-capacity section and the energy-capacity section based on the measured charge capacity. In examples, updating the first EOL counter includes converting the units of energy consumed to minutes of use.

### BRIEF DESCRIPTION OF THE DRAWINGS

The following drawing figures, which form a part of this application, are illustrative of aspects of systems and methods described below and are not meant to limit the scope of the disclosure in any manner, which scope shall be based on the claims.

FIGS. 1A-1B depict views of a video laryngoscope with a battery pack removed.

FIG. 2 depicts an example battery pack for video laryngoscopes.

FIG. 3 depicts voltages that may be supplied by a battery pack for video laryngoscopes.

FIG. 4 depicts an example method that may be performed by a battery pack for video laryngoscopes.

FIG. 5 depicts an example method that may be performed by a system that includes a battery pack for video laryngoscope.

FIG. 6A depicts example memory sections for the RFID tag of the battery pack.

FIG. 6B depicts an example method performed by a system including a battery pack and a first type of video laryngoscope.

FIG. 6C depicts an example method performed by a system including the battery pack and a second type of video laryngoscope.

FIG. 6D depicts an example method performed by the battery pack when being charged.

### DETAILED DESCRIPTION

Portable medical devices such as video laryngoscopes may use a battery pack to avoid the need for power cables. As an illustrative example, a video laryngoscope typically includes a handle that is held by the clinician and used to guide a tube with a video camera into the patient, thereby providing the clinician with better visibility during a procedure such as an intubation. A battery pack may be installed in the handle of the laryngoscope to power the laryngoscope. Other medical devices may also have handles (or other body portions) into which a battery pack may be installed.

For patient safety, it is often important that a battery installed in a medical device has sufficient charge such that the medical device maintains power throughout critical medical procedures. In a video laryngoscope, for example, a battery pack should have sufficient charge to provide power to the laryngoscope throughout an intubation procedure. The length of time that the battery can provide power to the medical device depends on the state of charge of the battery and the power consumption of the medical device, the latter of which may differ depending on the type of medical device used. For example, different video laryngoscopes may have different power consumption characteristics and thus the same amount of battery charge may provide different durations of power. It is desirable for the clinician to be able to monitor the remaining capacity and/or remaining minutes of the battery during a procedure.

In addition, it may be desirable to monitor a total lifetime usage time for a battery pack to ensure that the battery pack is not used beyond a predetermined maximum lifetime usage. Such tracking may be referred to as end-of-life (EOL) tracking.

As described herein, a battery pack includes a battery, a radio-frequency identification (RFID) tag, a capacity gauge, a voltage regulator, and a processor. In some examples, the battery pack is configured to, while installed in a medical device and providing power, periodically determine a remaining capacity of the battery (e.g., in milliamp hours (mAh) or other units) and store a value representing the remaining capacity in a register of the RFID tag. The medical device in which the battery pack is installed includes an RFID tag reader and can read the value in the RFID tag. The medical device may convert the remaining capacity value to a remaining minutes value based on a power consumption value associated with the medical device. The medical device may display the remaining capacity value and/or the remaining minutes value for viewing by a clinician. As an illustrative example, the medical device may be a video laryngoscope that has a camera at the distal end and a display screen that displays the camera feed during an intubation procedure (or other type of procedure). The video laryngoscope may display the remaining capacity and/or remaining minutes on the display screen to notify the clinician of the battery's status.

In some examples, a battery pack is configured to be installed in multiple different types of medical devices (e.g., in multiple different types of video laryngoscopes) that may have differing capabilities and power consumption. Thus, the battery pack may be configured to identify the type of medical device into which it is installed and perform different computations and/or operations based on the type of medical device.

For example, when the battery pack is installed in a first type of medical device, the battery pack may maintain an end-of-life (EOL) counter that represents the total number of minutes that the medical device has been used over its lifetime by periodically (e.g., every minute, or with another periodicity) incrementing a counter (e.g., starting from 0) or decrementing the counter (e.g., starting from a predetermined EOL value, such as 10,000) to represent an additional minute of use. In some examples, the battery pack maintains an EOL counter in a register of the RFID tag. The EOL counter represents the lifetime usage of the medical device and is a safety feature to ensure that the medical device and/or battery pack are not used past the point at which they may experience malfunctions.

In some examples, when the battery pack is installed in a second type of medical device, the medical device periodically writes a value to an EOL counter of the RFID tag on the battery pack to update the EOL counter. That is, the medical device causes the battery pack to update the value of the EOL counter rather than (or in addition to) the battery pack determining when to update this value and/or how much the value should be incremented. In some examples, the medical device writes the EOL value by activating an inductive metal coil that provides a signal (e.g., an electromagnetic field) to the RFID tag of the battery pack and causes the RFID tag to store the EOL value in a register.

Thus, the battery pack maintains an EOL counter independently and/or in response to signals received from the medical device, depending on the type of medical device in which the battery pack is installed.

In some examples, the battery pack includes multiple different EOL counters (e.g., in multiple registers of the RFID tag), each of which is associated with a different type of medical device (or group of medical devices) into which the battery may be installed. For example, the battery pack may include a first EOL counter that tracks minutes during which the battery pack is active in (e.g., providing power to) a first type of medical device, and a second EOL counter that tracks minutes during which the battery pack is active in a second type of medical device. In some cases, the battery pack updates the first EOL counter and the medical device updates the second EOL counter by providing a signal to the RFID tag on the battery pack, as described above.

The battery pack may also supply a different voltage to the medical device depending on the type of the medical device. For example, the battery pack may supply a first voltage to a first type of medical device and a second voltage to a second type of medical device, based on a determination, by the battery pack, of the type of medical device.

Additional details are now provided by way of discussion of the included drawings. While examples herein are directed to battery packs for video laryngoscopes, it should be appreciated that the same or similar techniques can be used in other types of battery-powered devices without departing from the scope of the disclosure.

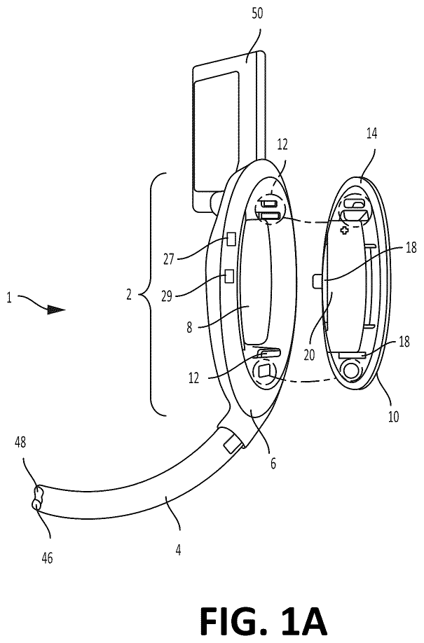

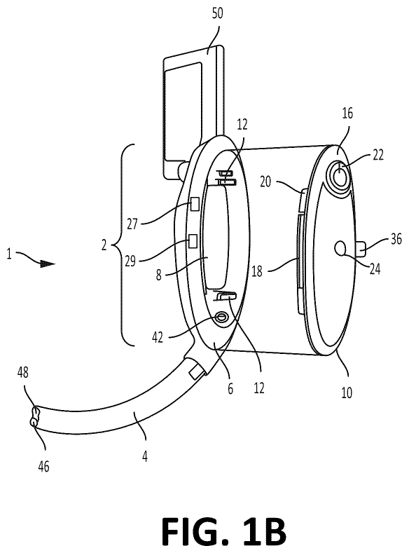

FIGS. 1A and 1B depict views of a video laryngoscope 1 (an example of a medical device) with a battery pack 10 that is not yet installed in the laryngoscope 1. The laryngoscope 1 includes a handle 2 and an arm 4 extending from the body. When prepared for use, a disposable rigid plastic sheath (not shown) is demountably retained on the arm, functioning as a laryngoscope blade that is inserted into the patient. The handle 2 of the laryngoscope includes a processor 29 that controls the functions and hardware of the laryngoscope, including controlling a light, a video camera, a video display for displaying images from the video camera, and an RFID tag reader 27 in the handle 2. The handle 2 comprises a first surface region 6 and a recess 8 for receiving a battery pack 10.

The handle 2 includes electrical contacts 12 (functioning as an input interface) for receiving electrical power from the battery pack, located within the recess. The battery pack includes a first major surface 14 and a second, opposed major surface 16 (shown in FIG. 1B). The first major surface includes battery retaining formations 18 which retain a battery pack 20. When the battery pack is installed on the handle 2 the first major surface faces the handle 2. The second major surface of the battery pack 20 includes an activation switch 22 and a light emitting diode 24 which provides an indication of a charge status and/or remaining capacity status of the battery. When the battery pack 20 is installed on the handle 2, the second major surface becomes a part of the outer surface of the handle, forming a grip for a user, along with the first surface region 6. The battery pack 20 comprises electrical contacts 26 (functioning as an output interface) through which power can be transmitted to the electrical contacts on the body of the medical device when the battery pack 20 is fitted and the device is powered on.

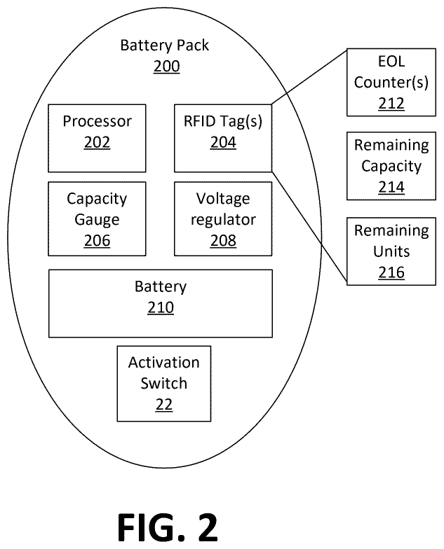

FIG. 2 depicts an example of a battery pack 200 that can be installed in a handle of a laryngoscope (e.g., a video laryngoscope such as shown in FIG. 1). The battery pack 200 includes a processor 202 (e.g., a microcontroller, microprocessor, application-specific IC, or other processing circuitry), an RFID tag 204, a capacity gauge 206, a voltage regulator 208, an activation switch 22 (as described with reference to FIGS. 1A-1B) and a battery 210. The battery 210 may be an alkaline, NiMH, or lithium battery, for example. The battery 210 may also be rechargeable.

The RFID tag 204 includes an RFID antenna (such as a coil in which a current is induced) for transmitting and/or receiving information and/or receiving power via radio-frequency electromagnetic waves. The RFID tag 204 also includes memory in which information can be stored. The RFID tag 204 may be powered (e.g., may include a battery) or may be a passive (unpowered) RFID tag 204, such as a near-field communication (NFC) tag that can be powered by electromagnetic fields emitted by an RFID tag reader. As mentioned with reference to FIG. 1, a laryngoscope in which the battery pack is installed may include an RFID tag reader (e.g., including an inductive coil) capable of inductively communicating with the RFID tag 204 when the battery pack is fitted to the laryngoscope, to read data from the RFID tag 204 memory and/or to write data to the memory.

The RFID tag 204 may include one or more registers (e.g., portions of the memory) that are used for maintaining a remaining capacity value 214, a remaining units value 216, and/or one or more EOL counters 212. The remaining capacity value 214 provides an indication of the remaining capacity of the battery 210 in units of mAh and is periodically updated by the battery pack based on measurements of the battery capacity by the capacity gauge 206. The remaining units value 216 also provides an indication of the remaining capacity of the battery but is not based on direct measurements of the battery capacity (e.g., charge measurements). Instead, remaining units value 216 may be updated during a procedure and/or at power-down of the battery pack or medical device by decreasing an initial fixed capacity value associated with the battery (e.g., a capacity value that is stored in memory rather than measured). This value is updated based on an elapsed operating time and a known (e.g., stored) power consumption value for the handle. The remaining capacity value 214 and remaining units value 216 are different types of battery capacity values.

The EOL counter(s) 212 provide an indication of how many minutes are remaining (or how many minutes have been consumed) in the lifetime of the laryngoscope and/or the battery, which may be based on incrementing an initial value or decrementing a predetermined initial value (such as 10,000 minutes) that is associated with a maximum amount of time the laryngoscope and/or battery should be used before they are at risk of malfunctions.

Operation of the video laryngoscope begins upon a pressing (e.g., by a user) of an activation switch 22 on the battery pack 200, causing the battery pack 200 to power up. Upon power up, the battery pack 200 may determine a remaining capacity (e.g., in mAh) of the battery using the capacity gauge 206. The capacity gauge 206 (sometimes referred to as a battery fuel gauge) may be configured to determine (e.g., measure or monitor) voltages, currents, temperatures, and/or impedances associated with the battery pack 200 during use and/or at start up to estimate a charge capacity of the battery.

The battery pack 200 provides a power-on signal to the handle of the laryngoscope (e.g., to the medical device) to cause the handle to power up. As discussed in more detail later, in some examples, the battery pack 200 provides the power-on signal at a different time (e.g., after a different delay) after powering up, and/or provides a different voltage, depending on the type of the medical device. In some examples, in response to detecting an activation of the activation switch (e.g., a power-up condition), the battery pack 200 determines whether a remaining capacity of the battery is greater than a threshold value. If the remaining capacity is greater than the threshold value, the battery pack enables its power output to supply power to the medical device. If the remaining capacity is not greater than the threshold value, the battery pack disables its power output (e.g., so that the medical device cannot begin a procedure that is unlikely to be completed with the battery). In some examples, if the medical device determines that the remaining minutes value fails to satisfy a threshold, the medical device disables the battery pack.

Upon power up, the battery pack 200 may write the remaining capacity value to a first register of the RFID tag 204. In some examples, the battery pack 200 writes the remaining capacity value to the RFID tag using an inductive coil in the battery pack 200 that powers up the RFID tag and writes the value (e.g., if the RFID tag is a passive RFID tag). In some examples, the battery pack writes the remaining capacity value to the RFID tag using instructions executed by the processor (e.g., if the RFID tag is an active RFID tag). In some examples, while powered on and/or installed in the handle, the battery pack updates the remaining capacity value periodically, such as every 1, 5, 10, 30, 60, 120, or 360 seconds. In some examples, the battery pack updates the remaining capacity value periodically while being charged (e.g., while installed in a charging station and receiving charge). The battery pack updates the remaining capacity value by saving a new value to the appropriate register in the RFID tag.

In some examples, if the battery determines that the remaining capacity satisfies a threshold (e.g., is greater than or equal to a minimum value, such as 30 mAh) the battery pack enables its power output (e.g., to supply power to a medical device) upon detecting a press of the activation switch 22. If the battery determines that the remaining capacity fails to satisfy the threshold (e.g., is less than a minimum value), the battery pack powers down to protect the battery from over-discharge and to keep a buffer capacity for shelf life and to await a next recharge cycle.

In some examples, when a first threshold value of an EOL counter 212 (or the sum of EOL counters) is satisfied, the battery pack transitions to an end-of-life warning mode in which the battery LED lights up alternating purple when in the charger to indicate to the user that the battery is close to end-of-life. For instance, when the total minutes used for the battery exceeds 9,500 minutes, and end-of life warning mode may be entered. The purple indicator light may be in addition to red, yellow, and green charging indicator lights that indicate whether the battery state of charge is low, medium, or full, respectively. When a second threshold value of the EOL counter 212 is reached, the battery pack transitions to an end-of-life mode where recharging is disabled.

An inductive coil in the laryngoscope handle (e.g., handle 2) may activate the RFID tag (if it is passive) and/or read the first register to obtain the remaining capacity. In some examples, the handle determines a remaining minutes value based on the remaining capacity value and on a predetermined power consumption of the laryngoscope. The predetermined power consumption may be a power consumption value that is stored on the handle, for example. In some examples, the handle may identify a first predetermined power consumption value if the handle is operating in a first mode (e.g., with the wireless communication or streaming features activated) and a second pre-determined power consumption if the handle is operating in a second mode (e.g., with the wireless communication or streaming features deactivated). In some examples, if the handle determines that the remaining minutes and/or remaining capacity are insufficient to perform a full procedure (e.g., the value fails to satisfy a threshold, such as by being below a predetermined minimum value), the handle may power itself off. Once the remaining capacity of the battery pack reaches a threshold value, one or more functions of the laryngoscope may stop operating or no longer start operating. The latter option may be preferable for a video laryngoscope used in an emergency environments as it could be dangerous for the device to stop functioning during an emergency procedure. It would be safer for it simply to not start functioning unless there was sufficient remaining capacity to complete a procedure based on the known typical power consumption during that procedure.

In some examples, in response to (e.g., within 1 second of) powering up, the battery pack (e.g., the processor 202) determines the type of the handle in which it is installed. The battery pack 200 may make this determination in various ways. For example, different handles may have different startup voltage demand timings (e.g., delays), which can be detected by the battery pack 200. In some examples, the battery pack 200 performs a handle load measurement to measure the electrical load on the battery 210, which may vary depending on the handle. The battery pack 200 may use the measured electrical load and/or startup voltage timing to determine the type of the handle.

In some examples, the battery pack 200 performs different operations depending on the type of the handle. For example, the battery pack may maintain one or more EOL counters 212 in different ways depending on the type of handle in which the battery pack 200 is installed (as determined by the battery pack 200). For example, either the battery pack 200 or the handle may be configured to determine when to update an EOL counter 212, depending on the type of handle in which the battery pack is installed.

In accordance with a determination that the battery pack is installed in a first type of handle, the battery pack maintains an EOL counter 212 itself by periodically updating (e.g., incrementing or decrementing) the EOL counter 212 (e.g., in accordance with a clock or timer on the battery pack or based on a periodic determination of the remaining capacity and a known power consumption of the handle). For example, the battery pack 200 may update the EOL counter every 1, 5, 10, 30, 60, 120, or 360 seconds. In accordance with a determination that the battery pack is installed in a second type of handle, the battery pack updates an EOL counter 212 in response to receiving signals from the handle (e.g., rather than in response to its own determination). For example, the handle may periodically (e.g., every 1, 5, 10, 30, 60, 120, or 360 seconds) cause the inductive coil in the handle to power up the RFID tag in the battery pack and write a new value to the EOL counter 212 in the RFID tag, reflecting the reduced available minutes before end-of-life is reached. The handle may update the EOL counter 212 depending solely on the amount of time for which the laryngoscope has been used, or based on its duration of use in one or more operating modes (e.g. modes in which the wireless communication features are enabled/disabled).

In some examples, the battery pack 200 maintains multiple EOL counters 212 in the RFID tag 204 (or in multiple RFID tags 204). For example, when the battery pack 200 is installed in a first type of handle, the battery pack 200 may update a first EOL counter itself in accordance with a clock or timer on the battery pack and/or based on a periodic determination of the remaining capacity and a known power consumption of the handle (e.g., by tracking the consumed mAh and converting to minutes based on the known power consumption of the handle). When the battery pack 200 is installed in a second type of handle, the battery pack 200 may update a second EOL counter in response to signals received from the handle. In this case, the total amount of time the battery pack has been used is represented by the combination of the first EOL counter and the second EOL counter. In some examples, when the sum of these two counters is greater than or equal to a first threshold value (e.g., ≥9,400 minutes or another value) the battery pack 200 transitions to an end-of-life-warning mode, in which a notification may be displayed on the video display and/or an indicator light may be displayed during charging. When the battery pack exceeds a second threshold larger than the first threshold (e.g., 10,000 or another value), the battery pack 200 may transition to an end-of-life mode in which the battery cannot be recharged. The first threshold may be, for example, 70%, 80%, 90%, or 95% of the second threshold.

In some examples, the battery pack 200 provides a different voltage output to the handle depending on the type of handle in which the battery pack 200 is installed (as determined by the battery pack). Some medical devices may be limited in the magnitude and/or variation of the voltage received from the battery. For example, a medical device may require a particular fixed voltage supply that may be a lower voltage than the voltage the battery 210 actually supplies. In this case, the battery pack 200 may use a voltage regulator 208 to supply a fixed (regulated) voltage to the medical device. Use of the voltage regulator may reduce the energy efficiency of the battery pack, however, because the voltage regulator dissipates power to reduce the voltage. Other medical devices may be capable of receiving higher voltages, eliminating the need for the voltage regulator 208 and the associated energy loss. Thus, the battery pack 200 may be configured to bypass the voltage regulator 208 and provide a direct (unregulated) voltage output from the battery 210 when the battery pack 200 determines that it is installed in a second type of medical device. That is, in accordance with a determination that the type of the medical device is a first type, the battery pack provides, via the voltage regulator, a fixed voltage to the medical device. In accordance with a determination that the medical device is the second type of medical device, the battery pack provides a direct varying voltage from the battery to the medical device, bypassing the voltage regulator 208.

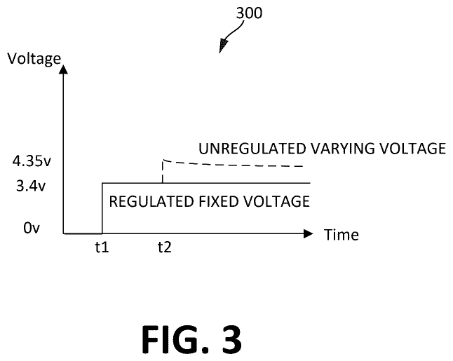

FIG. 3 depicts a plot 300 of different voltages that may be supplied by the battery pack, depending on the type of medical device detected by the battery pack. The plot 300 depicts voltage versus time of power delivered by the battery pack.

At time t1, the medical device and battery pack are powered up. At time t1 the battery pack has not yet determined the type of medical device in which it is installed, and thus the battery pack provides a first fixed voltage to the medical device (such as 3.4 volts, for example) via a voltage regulator. The first fixed voltage may be associated with a required supply voltage of a first type of medical device. At time t2, the battery pack determines the type of the medical device. If the medical device is the first type, the battery pack continues to supply (using the voltage regulator) the first fixed voltage to the medical device. If the battery pack determines that the medical device is the second type, the battery pack bypasses the voltage regulator and supplies a direct voltage from the battery to the medical device. Bypassing of the voltage regulator may be performed via a switch (e.g., transistor, relay) and/or logic executed by the processor. This bypassed voltage may start at a higher voltage than the fixed voltage, such as (for example) 4.35 volts, and may gradually decrease over time (e.g., to 3.0 volts or another voltage). In this manner, the battery pack may improve the energy efficiency and performance of medical devices that are capable of receiving higher and/or variable voltages, while maintaining compatibility with medical device that are configured to receive a lower and/or fixed voltage.

In the examples of FIGS. 1A-2, the battery pack powers up in response to the user pressing an activation switch on the battery pack (e.g., activation switch 22). In some examples, the battery pack subsequently provides a power-on signal (e.g., a voltage) to the laryngoscope to cause the laryngoscope to power up. In some examples, the power-on signal and/or the timing at which the battery pack provides the power-on signal to the laryngoscope depends on the laryngoscope in which the battery pack is installed, to accommodate differences in power-on logic for different laryngoscopes. For example, in accordance with a determination that a medical device is a first type of medical device, the battery pack provides a first power-on signal to the medical device at a first time. In accordance with a determination that the medical device is a second type of medical device, the battery pack provides a second power-on signal to the medical device at a second time. The first power-on signal and the second power-on signal may be the same or different signals.

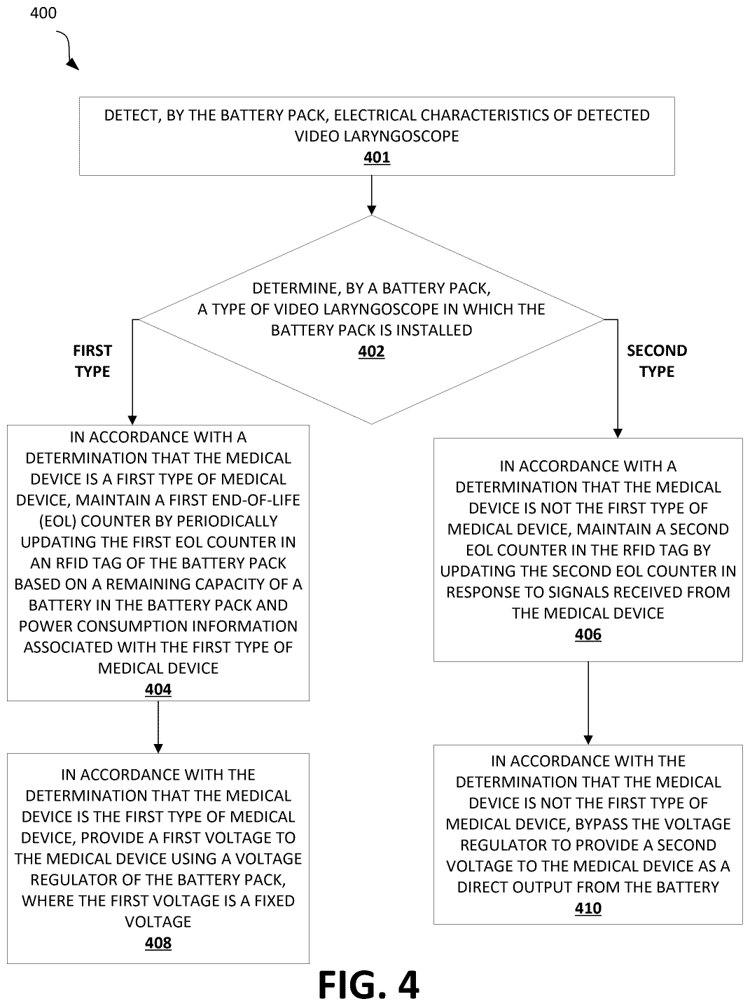

FIG. 4 depicts an example method 400 that may be performed by a battery pack (e.g., battery pack 200) for video laryngoscopes.

At operation 401, the battery pack determines one or more electrical characteristics of the video laryngoscope to which the battery pack is connected. The characteristics may include the electrical load of the video laryngoscope, such as voltage, current, and/or impedance.

At operation 402, the battery pack determines a type of video laryngoscope in which the battery pack is installed. For example, the battery pack determines the type of medical device based on an electrical characteristic of the medical device, such as an amount of electrical load of the medical device and/or a timing of an electrical load of the medical device. For example, the battery pack may measure an electrical load (e.g., a voltage and/or current) of the video laryngoscope in operation 401 and determine in operation 402 whether the electrical load exceeds a threshold electrical load. In accordance with a determination the that electrical load does not exceed the threshold electrical load, the battery back determines that the type of the video laryngoscope is a first type of video laryngoscope. In accordance with a determination the that electrical load exceeds the threshold electrical load, the battery back determines that the type of the video laryngoscope is a second type of video laryngoscope.

The battery pack then performs operation 404 or operation 406 as follows. At operation 404, in accordance with a determination that the medical device is a first type of video laryngoscope, at 404 the battery pack maintains a first EOL counter by periodically updating the first EOL counter in an RFID tag of the battery pack based on a remaining capacity of a battery in the battery pack and power consumption information associated with the first type of medical device.

At operation 406, in accordance with a determination that the medical device is not the first type of medical device, at 406 the battery pack maintains a second EOL counter in the RFID tag by updating the second EOL counter in response to signals received from the medical device. For example, the RFID tag of the battery pack updates the second EOL counter in a second register of the RFID tag in response to receiving an electromagnetic signal (e.g., being exposed to an electromagnetic field) from an RFID tag reader/writer of the video laryngoscope.

Additionally or alternatively, the battery pack performs operation 408 or operation 410 as follows. At operation 408, in accordance with the determination that the medical device is the first type of medical device, the battery pack provides a first voltage to the medical device using a voltage regulator of the battery pack, where the first voltage is a fixed voltage. The first voltage may be a voltage associated with the first type of medical device, and the first voltage may be a lower voltage than the voltage supplied directly by the battery.

At operation 410, in accordance with the determination that the medical device is not the first type of medical device, the battery pack bypasses the voltage regulator to provide a second voltage to the medical device as a direct output from the battery. The second voltage may initially be higher than the first voltage and may decrease over time.

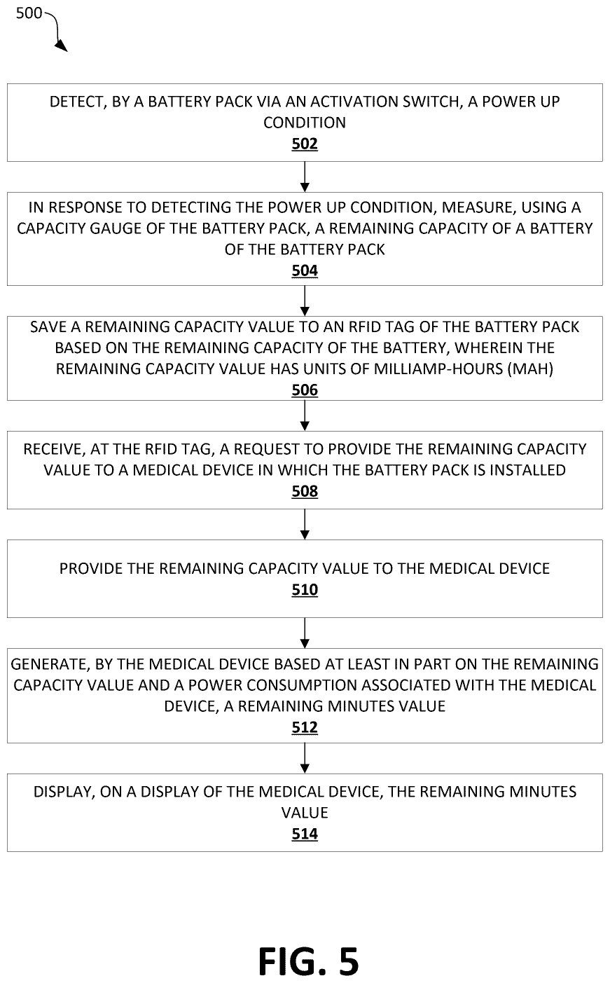

FIG. 5 depicts an example method 500 that may be performed by a system that includes a video laryngoscope (e.g., laryngoscope 1) and a battery pack (e.g., battery pack 200). At operation 502, the battery pack detects, via an activation switch (e.g., activation switch 22), a power up condition. For example, the battery pack detects that the activation switch has been pressed. At operation 504, in response to detecting the power up condition, the battery pack measures, using a capacity gauge of the battery pack, a remaining capacity of a battery of the battery pack. At operation 506, the battery pack saves a remaining capacity value to an RFID tag of the battery pack based on the remaining capacity of the battery, wherein the remaining capacity value has units of milliamp-hours (mAh).

At operation 508, the battery pack receives, at the RFID tag, a request to provide the remaining capacity value to the medical device in which the battery pack is installed. For example, the battery pack receives the request by receiving an electromagnetic signal (e.g., a field) from an RFID tag reader of the medical device that causes the RFID tag to power up and receive the request from the RFID tag reader. At operation 510, the battery pack provides the remaining capacity value to the medical device, such as by transmitting (e.g., radiating) an electromagnetic field from the RFID tag that includes the remaining capacity value.

At operation 512, the medical device generates, based at least in part on the remaining capacity value and a power consumption associated with the medical device, a remaining minutes value. For example, the medical device divides the remaining capacity value by the power consumption (e.g., the electrical current draw of the medical device) to obtain the remaining minutes value. At operation 514, the medical device displays, on a display of the medical device, the remaining minutes value.

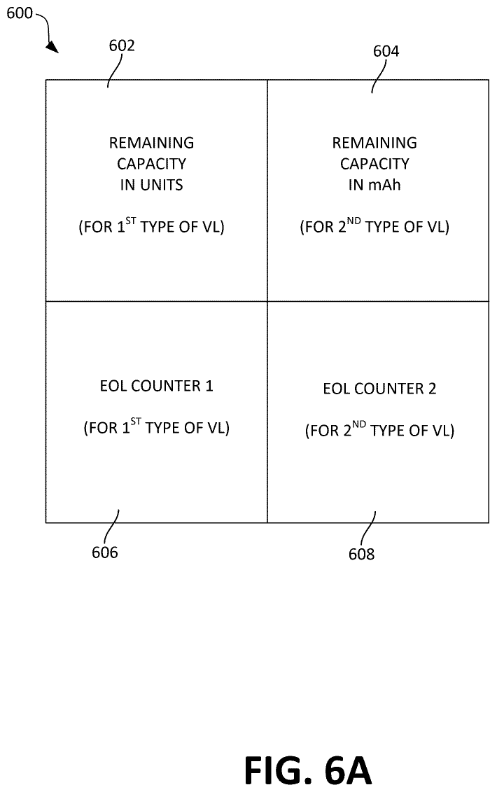

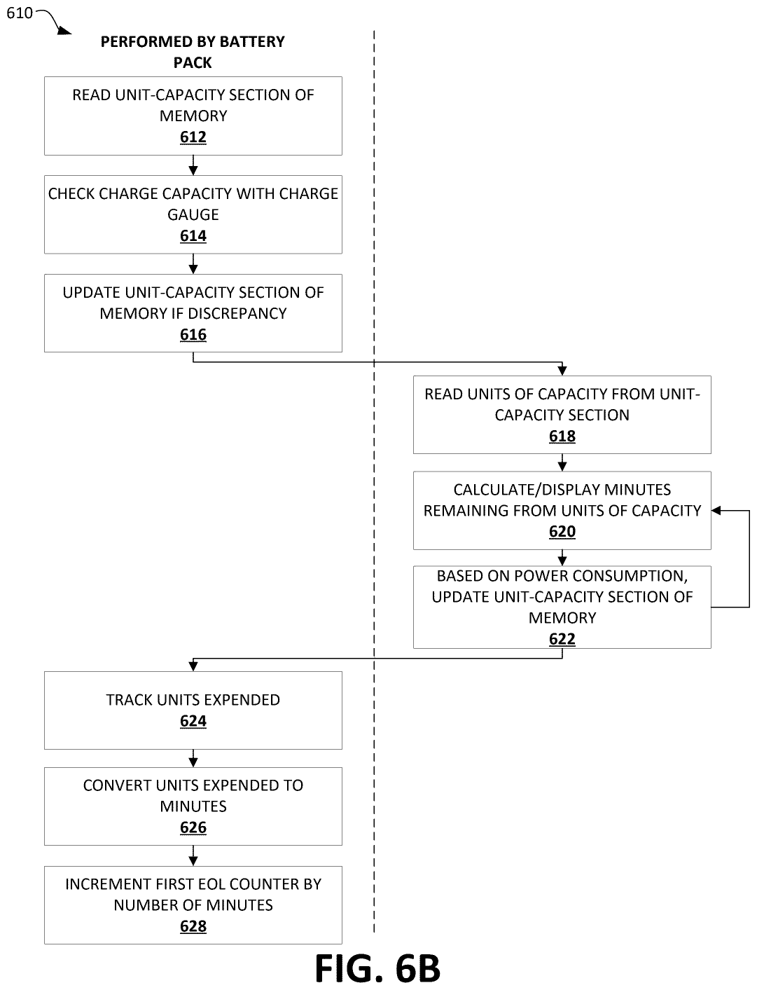

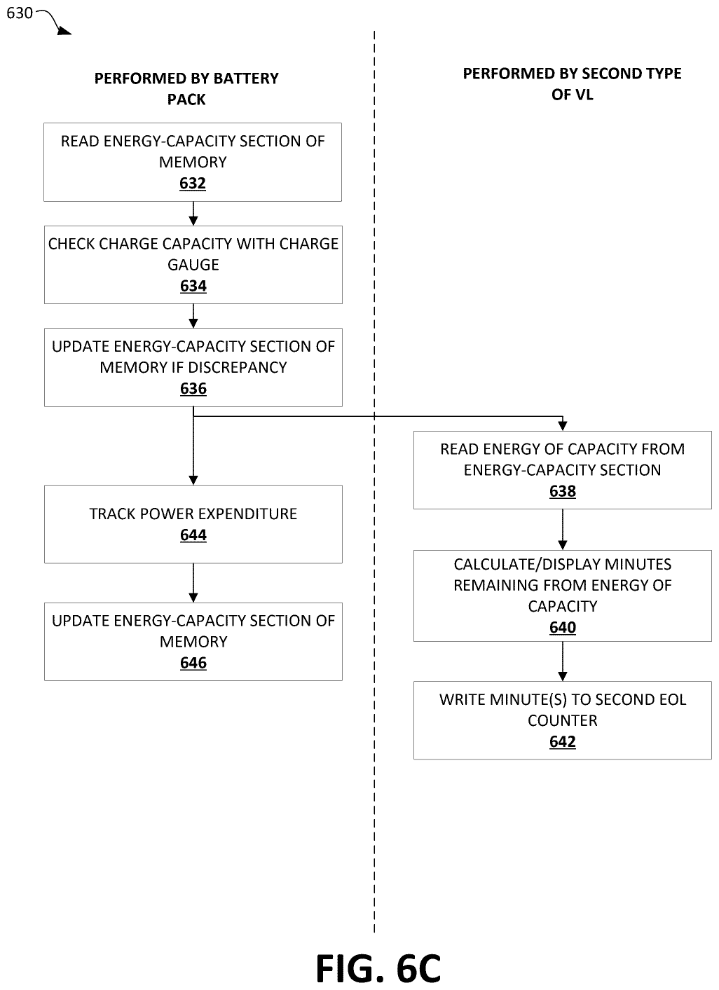

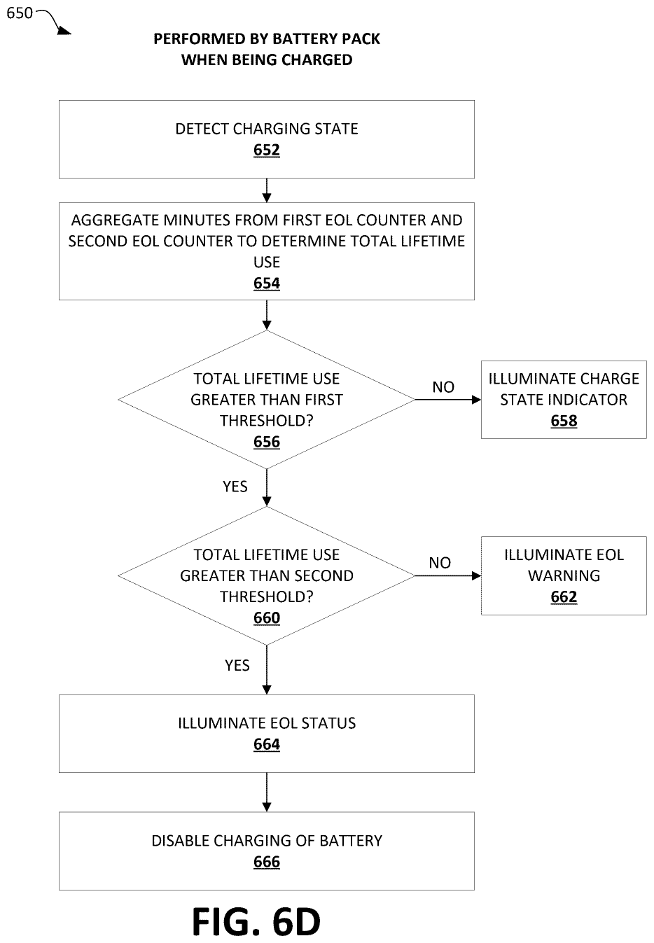

FIG. 6A depicts example memory sections 600 for the RFID tag of the battery pack. As described above, the battery pack may be used with different types of video laryngoscopes that have different capabilities that may result in different power consumption. To allow for such interoperability, the memory sections of the RFID tag of the battery pack include different sections for data from the respective types of video laryngoscopes. For instance, the battery pack stores the remaining capacity of the battery in two different sections-namely a unit-capacity section 602 and an energy-capacity section 604. The unit-capacity section 602 stores a representation of the remaining charge capacity in “units” of charge capacity remaining (which may be a unitless or arbitrary value representing charge capacity). The energy-capacity section 604 stores a representation of the remaining charge capacity in an energy representation, such as mAh. The unit-capacity section 602 may be used to provide charge capacity data to the first type of video laryngoscope, and the energy-capacity section 604 may be used to provide charge capacity data to the second type of video laryngoscope.

The battery pack stores the total lifetime usage data in two different memory sections-namely a first EOL counter 606 and a second EOL counter 608. Both the first EOL counter 606 and the second EOL counter 608 store the lifetime usage data in temporal units, such as minutes. The sum or aggregation of the first EOL counter 606 and the second EOL counter 608 represents the total lifetime usage of the battery pack. The first EOL counter 606 stores usage of the battery pack with video laryngoscopes of the first type, and the second EOL counter 608 stores usage of the battery pack with video laryngoscopes of the second type.

When the battery pack is connected to a particular laryngoscope, the battery pack determines the type of battery pack. The different memory sections 600 may then be read or written to based on the type of video laryngoscope.

FIG. 6B depicts an example method 610 performed by a system including the battery pack and a first type of video laryngoscope. The method may be performed after or concurrently with method 400 in FIG. 4, including detecting a type of video laryngoscope and providing the appropriate voltage.

At operation 612, the battery pack reads the unit-capacity section 602 to receive the stored value. At operation 614, the battery pack checks the charge capacity of the battery based on the charge gauge readings. If there is a discrepancy between the value in the unit-capacity section 602 and the charge capacity based on the charge gauge readings, the unit-capacity section 602 is overwritten with the charge gauge reading at operation 616.

At operation 618, once the first type of the video laryngoscope has powered, the first type of video laryngoscope reads the units of capacity from the unit-capacity section 602. This may be performed by a wireless RFID read operation from the RFID tag of the battery pack. At operation 620, the first type of video laryngoscope calculates the number of minutes remaining based on the units of capacity read in operation 618. The first type of video laryngoscope may then display the remaining minutes of capacity as part of operation 618. At operation 622, based on the power consumption of the video laryngoscope, the video laryngoscope updates the unit-capacity section 602 of the battery pack. For instance, the video laryngoscope transmits a write command via an RFID signal to the RFID tag of the battery pack to cause the unit-capacity section 602 to be updated. Operations 620-622 may repeat such that the video laryngoscope continues to calculate the remaining minutes and update the unit-capacity section 602 to reflect the units of energy consumed from the battery pack by the video laryngoscope. For example, operations 620-622 may repeat every minute (or other interval).

At operation 624, the battery pack determines how many units of energy have been expended by the battery pack. Because the battery pack knows the power draw of the first type of video laryngoscope (e.g., has a power consumption value stored in memory), the battery pack is then able convert the units of energy expended to a number of minutes that the battery pack has been in use. This conversion of units expended to minutes of use is performed in operation 626. At operation 628, the first EOL counter 606 is incremented by the number of minutes of use calculated at operation 626. Operations 624-628 may be performed as the units of energy in the unit-capacity section 602 are updated. For instance, the first EOL counter 606 may be updated each time the unit-capacity section 602 is updated. In other examples, operations 624-628 may be performed as part of shut down routine when the video laryngoscope is powered off. For instance, the first EOL counter 606 may be updated for the total number of units of energy expended by the battery pack during the duration that the video laryngoscope was powered on.

The method 610 may then also repeat each time that the battery pack is connected to the first type of video laryngoscope (and powered on). Accordingly, for each use of the battery pack with the first type of video laryngoscope, the unit-capacity section 602 is incremented for the number of minutes that the battery pack was in use providing power to the first type of video laryngoscope.

FIG. 6C depicts an example method 630 performed by a system including the battery pack and a second type of video laryngoscope. The method may be performed after or concurrently with method 400 in FIG. 4, including detecting a type of video laryngoscope and providing the appropriate voltage.

Due to the second type of video laryngoscope being used, method 630 differs from method 610 in how the remaining energy of the battery pack is tracked and how total minutes of usage is tracked. For example, the energy-capacity section 604 and the second EOL counter 608 are used instead of the unit-capacity section 602 and the first EOL counter 606. The differences in operations are performed in part because the second type of video laryngoscope may have a different power consumption than the first type of video laryngoscope and that power consumption may also be variable. Accordingly, a minute of use by the second type of video laryngoscope differs in energy consumption than a minute of use by the first type of video laryngoscope. Nevertheless, the usage with both the first type and second type of video laryngoscope is tracked in minutes of use (or other temporal unit such as seconds, hours, etc.).

At operation 632, the battery pack reads the energy-capacity section 604 to receive the stored value. At operation 634, the battery pack checks the charge capacity of the battery based on the charge gauge readings. If there is a discrepancy between the value in the energy-capacity section 604 and the charge capacity based on the charge gauge readings, the energy-capacity section 604 is overwritten with the charge gauge reading at operation 636.

At operation 638, the second type of video laryngoscope reads the energy capacity of the battery pack from the energy-capacity section 604. For instance, the second type of video laryngoscope sends a wireless read request to the RFID tag of the battery pack to receive the mAh value in the energy-capacity section 604. At operation 640, the second type of video laryngoscope calculates the minutes remaining for use based on the energy capacity value read from the energy-capacity section 604. The second type of video laryngoscope may also display the remaining minutes remaining on a display screen of the second type of video laryngoscope. At operation 642, the video laryngoscope writes the number of minutes of use for the battery pack to the second EOL counter 608. For instance, the video laryngoscope transmits a wireless write function to the RFID tag of the battery pack. In examples, operation 642 is performed every minute of use. Accordingly, the second EOL counter 608 is incremented by one minute for every minute of use. In other examples, operation 642 may be performed to increment the second EOL counter 608 at different intervals.

At operation 644, the power expenditure by the battery pack is tracked or measured by the battery back, such as by the capacity gauge. At operation 646, the battery pack updates the energy-capacity section 604 based on the power expenditure detected in operation 644. Accordingly, with the second type of the video laryngoscope, the battery pack tracks its own power expenditure and remaining charge capacity. The second type of video laryngoscope, however, tracks the minutes of use and updates the second EOL counter 608.

FIG. 6D depicts an example method 650 performed by the battery pack when being charged. For instance, when the battery pack is inserted into a charging device and/or connecting to a charging power supply, the operations of method 650 may be performed.

At operation 652, the battery pack detects that it is in a charging state. Such a detection may be determined from energy flowing into the battery pack from either a wired or wireless source. At operation 654, battery pack aggregates the minutes in the first EOL counter 606 and the minutes in the second EOL counter 608 to determine the total lifetime use of the battery pack.

At operation 656, the battery pack determines if the total lifetime use is greater than a first threshold. The first threshold indicates when an EOL warning state should be entered. For example, when the battery pack is designed for 10,000 minutes of total use, the first threshold is a number less than 10,000 minutes, such as 9,400 minutes.

If the total lifetime use is less than the first threshold, the method 650 flows to operation 658 where a charge state indicator of the battery back is illuminated. For instance, the charge state indicator may be a particular color of light that is illuminated based on the charge state of the battery pack while charging. A low charge state may be associated with the color red, a medium charge state may be associated with the color yellow, and a full charge state may be associated with the color green.

If the total lifetimes use is greater than or equal to the first threshold, the method 650 flows to operation 660 a determination is made if the total lifetime use is greater than a second threshold. The second threshold is the EOL threshold, such as 10,000 minutes of total lifetime use. If the total lifetime use is less than the second threshold (e.g., the total lifetime use is between the first threshold and the second threshold), the method flows to operation 6662 where the battery back illuminates an EOL warning. The EOL warning indicates that the battery pack is nearing its end of life. The EOL warning may be illuminated through the same light source as the charge state indicator illuminated in operation 658. In some examples the EOL warning illuminates a different color than the charge state indicators (such as purple). To indicate the EOL warning and the charge state information, the illumination may alternate between the charge state color (e.g., green, yellow, red) and the EOL warning color (e.g., purple).

If the total lifetime use exceeds the second threshold at operation 660, the method flows to operation 664 where the battery back illuminates an EOL status. Illuminating the EOL status may include changing the light source to be a solid color that indicates that the battery pack is at the end of its lifetime, such as by illuminating a solid purple color (e.g., the same color used for the EOL warning in operation 662). When the total lifetime use exceeds the second threshold, the battery pack has exceeded its lifetime and should no longer be used. Thus, at operation 666, the battery may disable further charging of the battery and/or disable further use of the battery pack.

The method 610, 630, and 650 may be performed at different times relative to one another. For instance, at a first point in time when the battery pack is connected to the first type of video laryngoscope, method 610 is performed. Then, at a second point in time after the battery pack has been removed from the first type of video laryngoscope, the method 630 is performed. Subsequently, at a third point in time, the battery pack is then removed the second type of video laryngoscope, connected to or inserted into a charging device, and method 630 is performed. Different orders are also possible.

While the methods depicted in FIGS. 4-6 are primarily discussed with respect to video laryngoscopes, the methods discuss therein may be applied to other types of medical devices that may receive the battery pack.

Those skilled in the art will recognize that the methods and systems of the present disclosure may be implemented in many manners and as such are not to be limited by the foregoing aspects and examples. For instance, while the above examples are primarily discussed in relation to a video laryngoscope, such techniques may be used in other types of surgical or non-surgical instruments. Further, any number of the features of the different aspects described herein may be combined into single or multiple aspects, and alternate aspects having fewer than or more than all of the features herein described are possible. Functionality may also be, in whole or in part, distributed among multiple components, in manners now known or to become known. Further, as used herein and in the claims, the phrase “at least one of element A, element B, or element C” is intended to convey any of: element A, element B, element C, elements A and B, elements A and C, elements B and C, and elements A, B, and C.

Numerous other changes may be made which will readily suggest themselves to those skilled in the art and which are encompassed in the spirit of the disclosure and as defined in the appended claims. While various aspects have been described for purposes of this disclosure, various changes and modifications may be made which are well within the scope of the disclosure. Numerous other changes may be made which will readily suggest themselves to those skilled in the art and which are encompassed in the spirit of the disclosure and as defined in the claims.

What is claimed is:

1. A battery pack configured to be installed in a video laryngoscope to provide power to the video laryngoscope, the battery pack comprising:
a battery;
a radio-frequency identification (RFID) tag; and
a processor, wherein the processor is configured to cause the battery pack to:
determine a type of the video laryngoscope; and
perform one of:
in accordance with a determination that the video laryngoscope is a first type of video laryngoscope, maintain a first end-of-life (EOL) counter by periodically updating the first EOL counter in the RFID tag based on a remaining capacity of the battery and power consumption information associated with the first type of video laryngoscope; or
in accordance with a determination that the video laryngoscope is not the first type of video laryngoscope, maintain a second EOL counter by updating the second EOL counter in response to signals received from the video laryngoscope.

2. The battery pack of claim 1, further comprising a capacity gauge configured to measure the remaining capacity of the battery, wherein the processor is further configured to cause the battery to:
in response to detecting a power up condition:
determine, using the capacity gauge, the remaining capacity of the battery; and
save a remaining capacity value to the RFID tag based on the remaining capacity of the battery.

3. The battery pack of claim 2, wherein the battery pack is configured to:
periodically determine, using the capacity gauge, the remaining capacity; and
update the remaining capacity value based on the remaining capacity.

4. The battery pack of claim 1, wherein the battery pack includes a voltage regulator, and the processor is further configured to cause the battery pack to:
perform one of:
in accordance with the determination that the video laryngoscope is the first type of video laryngoscope, provide a first voltage to the video laryngoscope using the voltage regulator, wherein the first voltage is a fixed voltage; and
in accordance with the determination that the video laryngoscope is the second type of video laryngoscope, bypass the voltage regulator to provide a second voltage to the video laryngoscope as a direct output from the battery.

5. The battery pack of claim 1, wherein the processor is further configured to cause the battery pack to:
in response to powering on, perform one of:
in accordance with the determination that the video laryngoscope is a first type of video laryngoscope, provide a first power-on signal to the video laryngoscope at a first time; and
in accordance with the determination that the video laryngoscope is a second type of video laryngoscope, provide a second power-on signal to the video laryngoscope at a second time.

6. The battery pack of claim 1, wherein the remaining capacity is in units of milliamp hours.

7. The battery pack of claim 1, wherein processor is configured to cause the battery pack to determine the type of video laryngoscope by identifying an electrical characteristic of the video laryngoscope.

8. The battery pack of claim 1, wherein the processor is configured to cause the battery pack to disable a power output of the battery pack based on a determination that the first EOL counter, the second EOL counter, or a combination of the first EOL counter and the second EOL counter has reached a threshold.

9. A system, comprising:
a battery pack comprising a battery, a first processor, an RFID tag, a capacity gauge, an activation switch, wherein the battery pack performs operations comprising:
detecting, by the battery pack via the activation switch, a power up condition;
in response to detecting the power up condition, measuring, using the capacity gauge, a remaining capacity of the battery;
saving a remaining capacity value to the RFID tag based on the remaining capacity of the battery;
receiving, at the RFID tag, a request to provide the remaining capacity value to a video laryngoscope comprising a second processor and a radio-frequency identification (RFID) tag reader;
providing the remaining capacity value to the video laryngoscope;
wherein the video laryngoscope performs operations comprising:
generating, by the video laryngoscope based at least in part on the remaining capacity value and a power consumption associated with the video laryngoscope, a remaining minutes value; and
displaying, on a display of the video laryngoscope, the remaining minutes value.

10. The system of claim 9, wherein the operations performed by the video laryngoscope include: periodically sending, using the RFID tag reader, a signal to the RFID tag to cause the RFID tag to update an end-of-life (EOL) counter in the RFID tag.

11. The system of claim 9, wherein the operations of the video laryngoscope include:
periodically obtaining an updated remaining capacity value from the RFID tag, updating the remaining minutes value based on the updated remaining capacity value, and
displaying the updated remaining minutes value.

12. The battery pack of claim 11, wherein the operations performed by the battery pack include:
periodically determining, using the capacity gauge, the remaining capacity; and
updating the remaining capacity value based on the remaining capacity.

13. The battery pack of claim 9, wherein the battery pack includes a voltage regulator, and the operations performed by the battery pack include:
determining that the video laryngoscope is a particular type of video laryngoscope; and
in accordance with the determination that the video laryngoscope is the particular type of video laryngoscope, providing a variable voltage to the video laryngoscope as an output from the battery pack.

14. The battery pack of claim 9, wherein the second processor is configured to cause the battery pack to determine that the video laryngoscope is a first type of video laryngoscope by:
measuring an electrical load of the video laryngoscope in milliamps, and determining that the electrical load exceeds a threshold electrical load.

15. A battery pack for a video laryngoscope, the battery pack comprising:
a radio-frequency identification (RFID) tag having access to a unit-capacity section, an energy-capacity section, a first end-of-life (EOL) counter, and a second EOL counter;
a processor; and
memory storing instructions that, when executed by the processor, cause the battery pack to perform operations comprising:
while connected to a first type of video laryngoscope:
receiving a read request, from the first type of video laryngoscope, for a charge capacity value from the unit-capacity section;
receiving an update, from the first type of video laryngoscope, to the unit-capacity section indicating units of energy consumed by the first type of video laryngoscope; and
based on the units of energy consumed by the first type of video laryngoscope, updating the first EOL counter; and
while connected to a second type of video laryngoscope:
receiving a read request, from the second type of video laryngoscope, for a charge capacity value from the energy-capacity section; and
receiving, from the second type of video laryngoscope, an update to the second EOL counter.

16. The battery pack of claim 15, wherein the operations further comprise:
while charging:
aggregating time of use from the first EOL counter and the second EOL counter to determine total lifetime use of the battery pack; and
based on total lifetime of use, indicating at least one of an EOL warning or an EOL status.

17. The battery pack of claim 16, wherein the EOL warning is indicated based on the total lifetime of use being between a first threshold and a second threshold, and the EOL warning indicates the battery is approaching end of life.

18. The battery pack of claim 17, wherein the EOL status is indicated based on the total lifetime of use being above the second threshold, wherein the EOL status indicates that the battery has reached its end of life.

19. The battery pack of claim 15, wherein the battery pack further comprises a charge gauge, and the operations further comprise:
measuring, by the charge gauge, a charge capacity of the battery pack; and
updating the unit-capacity section and the energy-capacity section based on the measured charge capacity.

20. The battery pack of claim 15, wherein updating the first EOL counter includes converting the units of energy consumed to minutes of use.
